(19) United States
(12) Patent Application Publication
(10) Pub. No.: US 2016/0287503 A1
(43) Pub. Date: Oct. 6, 2016

(54) MILD COSMETIC CLEANSING COMPOSITION

(71) Applicant: Henkel AG & Co. KGaA, Duesseldorf (DE)

(72) Inventor: Thomas Schroeder, Hamburg (DE)

(73) Assignee: Henkel AG & Co. KGaA, Duesseldorf (DE)

(21) Appl. No.: 15/175,934

(22) Filed: Jun. 7, 2016

Related U.S. Application Data
(63) Continuation of application No. PCT/EP2014/076728, filed on Dec. 5, 2014.

(30) Foreign Application Priority Data
Dec. 17, 2013 (DE) 10 2013 226 274.4

(51) Int. Cl. A61K 8/46; A61K 8/898; A61Q 19/00; A61K 8/81; A61Q 19/10; A61K 8/42; A61K 8/891

(52) U.S. Cl. CPC A61K 8/466; A61K 8/42; A61K 8/898; A61K 8/891; A61K 8/8152; A61Q 19/10; A61Q 19/007; A61K 2800/596

(57) ABSTRACT

A particularly mild, high-foaming cosmetic cleansing composition with care qualities includes, within an appropriate vehicle, a) at least one first anionic surfactant of the formula (1) described herein, and b) at least one second anionic surfactant of the formula (II) described herein.

### FIELD OF THE INVENTION

The present invention generally relates to cosmetics, and more particularly relates to mild cleansing agents that contain a special surfactant mixture.

### BACKGROUND OF THE INVENTION

Cosmetic cleansing agents, such as hair shampoos, are based on conventional anionic, amphoteric, zwitterionic, nonionic and/or cationic surfactants. Owing to their outstanding cleansing and foaming power, predominantly anionic surfactants, optionally mixed with small amounts of co-surfactants, are used. Typical anionic surfactants that are used in a multitude of commercially available shampoos are alkyl sulfates or alkyl ether sulfates. Alkyl ether sulfates are usually preferred since they are milder and have excellent foaming power.

When formulating particularly mild cleansing compositions for use on sensitive parts of the skin (such as the facial skin), for use on baby skin or for use on sensitive and/or damaged hair, alkyl ether sulfates are not always satisfactory since the irritation potential of the same is too high for these uses and they can impart increased brittleness to damaged hair.

Numerous attempts have therefore been made in the past to find particularly mild surfactant mixtures that have sufficiently large amounts of foam and sufficiently high foam qualities and that have little or no irritation potential on the skin and/or the mucous membrane. The surfactant mixtures additionally were to be suitable for use in hair treatment products, specifically for use in hair cleansing products having good care properties.

The application WO 92/08440 discloses mild surfactant mixtures having outstanding foam properties, which contain a mixture of acyl isethionates, zwitterionic surfactants, and alkyl ether sulfates. WO 11/015857 discloses cleansing compositions that have low irritation potential on the skin and contain novel C₅₋₃₀ alkoyl-alkyl isethionates and amphoteric surfactants in a weight ratio of 4:1 to 1:4. The mild cleansing agents are suitable for use as baby shampoo.

The disadvantage of many mild skin and hair cleansing agents is that the improved skin compatibility thereof often comes at the expense of the texture of the cleansing agents. The often unsatisfactory amounts of foam and foam properties that can be achieved with mild cleansing agents are another disadvantage. It has moreover been observed that the care properties of mild cleansing agents (in particular on hair) are not always satisfactory.

it is therefore desirable to produce particularly mild cleansing agents that are tolerated well by the skin and mucous membranes. The cleansing agents should have a user-friendly texture and, in conjunction with water, generate large amounts of foam. Furthermore, the cleansing agents should have improved care properties.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic cleansing agent includes in at least one suitable carrier at least one first anionic surfactant according to the following formula (I)

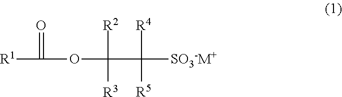

in which at least one of the groups R² to R⁵ denotes a C₁ to C₄ alkyl group, and the remaining groups independently of one another denote a hydrogen atom or a C₁ to C₄ alkyl group, R¹ in each case denotes a linear or branched, saturated or unsaturated alkyl group having 6 to 30 carbon atoms, and M⁺ in each case denotes an ammonium, alkanol ammonium or metal cation; at least one second anionic surfactant of the following formula (II)

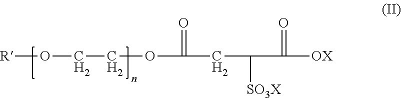

in which R′ denotes a linear or branched, saturated or unsaturated alkyl group having 6 to 30 carbon atoms, n denotes a number from 0 to 20, preferably from 1 to 10, and X denotes an ammonium, alkanol ammonium or metal cation; and at least one C₂ to C₄ monoalkanolamide and/or dialkanolamide of at least one C₈ to C₂₄ carboxylic acid.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject atter of the invention is a cosmetic cleansing agent, including in a suitable carrier
a) at least one first anionic surfactant according to the following formula (I)

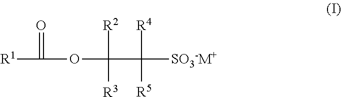

in which
at least one of the groups R² to R⁵ denotes a C₁ to C₄ alkyl group, and the remaining groups independently of one another denote a hydrogen atom or a C₁ to C₄ alkyl group;
R¹ in each case denotes a linear or branched, saturated or unsaturated alkyl group having 6 to 30 carbon atoms; and
M in each case denotes an ammonium, alkanol ammonium or metal cation;
b) at least one second anionic surfactant of the following formula (II)

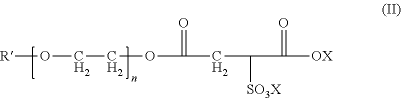

in which
R′ denotes a linear or branched, saturated or unsaturated alkyl group having 6 to 30 carbon atoms;
n denotes a number from 0 to 20, preferably from 1 to 10; and
X denotes an ammonium, alkanol ammonium or metal cation; and
c) at least one C₂ to C₄ monoalkanolamide and/or dialkanolamide of at least one C₈ to C₂₄ carboxylic acid.

A suitable carrier shall preferably be understood to mean an aqueous or aqueous-alcoholic carrier. The carrier preferably contains at least 50 wt. %, more preferably at least 60 wt. %, and particularly preferably at least 70 wt. % water.

The cosmetic carrier can moreover contain 0.01 to 40 wt. %, preferably 0.05 to 35 wt. %, and in particular 0.1 to 30 wt. % of at least one alcohol, which can be selected from ethanol, 1-propanol, 2-propanol, isopropanol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol_{;} 1,2-hexanediol, 1,6-hexandiol, polyethylene glycols, sorbitol, sorbitan, benzyl alcohol, phenoxyethanol or mixtures of these alcohols. The water-soluble alcohols are preferred. Ethanol, 1-propanol, 2-propanol, isopropanol, 1,2-propylene glycol, glycerol, benzyl alcohol and/or phenoxyethanol and mixtures of these alcohols are particularly preferred. In particular glycerol is preferred.

Preferred anionic surfactants of the aforementioned formula (I) comprise a linear or branched, saturated or unsaturated alky group having 8 to 18 carbon atoms as group R¹. The group R¹ particularly preferably denotes a C₈, C₁₀, C₁₁, C₁₄, C₁₆ group, or mixtures of these fatty acid groups, as they are obtained when the fatty acid(s) is/are derived from natural oils such as coconut oil.

In further preferred anionic surfactants of the aforementioned formula (I), the groups R² to R⁵ can each denote hydrogen or a methyl, ethyl, n-propyl, n-butyl or 2-butyl group. The groups R² to R⁵ preferably denote hydrogen, or at least one of the groups R² to R⁵ denotes a methyl, ethyl or n-propyl group, and in particular a methyl group. In a particularly preferred embodiment, one of the groups R² to R⁵ denotes a C₁ to C₄ alkyl group, in particular a methyl group, and the remaining groups each denote a hydrogen atom. In principle, it is also possible for the anionic surfactant according to formula (I) to contain a mixture of isomers including both components that comprise, for example, a C₁ to C₄ alkyl group, in particular a methyl group, as group R² and a hydrogen atom in each case as groups R³ to R⁵, and components that comprise, for example, a C₁ to C₄ alkyl group, in particular a methyl group, as group R⁵ and a hydrogen atom in each case as groups R² to R⁴. M⁺ in the aforementioned formula (I) preferably denotes an alkali metal cation or an ammonium ion. M⁺ particularly preferably denotes a potassium or a sodium ion, and in particular preferably a sodium ion. Particularly preferred anionic surfactants according to the aforementioned formula (I) are the compounds known by the MCI names Sodium Cocoyl isethionate, Sodium Lauroyl Isethionate, Sodium Myristyl isethionate, Sodium Palmitoyl Isethionate, Sodium Stearyl Isethionate, Sodium Oleyl Isethionate, Sodium Cocoyl Methyl Isethionate, Sodium Lauroyl Methyl Isethionate, Sodium Myristyl Methyl Isethionate, Sodium Palmitoyl Methyl Isethionate, Sodium Stearyl Methyl Isethionate and/or Sodium Oleyl Methyl isethionate. In particular, Sodium Cocoyl Methyl Isethionate and/or Sodium Lauroyl Methyl Isethionate are preferred. Corresponding commercial products are available, for example, from Innospec under the trade name “Iselux®” and from Clariant or Uniquema under the trade name “Hostapon®” or “Arlatone®”.

Preferred anionic surfactants of the aforementioned formula (II) preferably comprise a linear or branched, saturated or unsaturated alky group having 8 to 18 carbon atoms as the group R′ The group R′ particularly preferably denotes a C₈, C₁₀, C₁₂, C₁₄, C₁₆ group, or mixtures of these fatty acid groups, as they are obtained when the fatty acid(s) is/are derived from natural oils such as coconut oil. The number n particularly preferably denotes 0 or the numbers 1, 2, 3, 4, 5, 6, 7, or 8. X preferably denotes an alkali metal ion or an ammonium ion in the aforementioned formula (IV). X particularly preferably denotes a potassium or sodium ion, and in particular preferably a sodium ion.

Most particularly preferred anionic surfactants according to formula (II) are the compounds known by the INCI names Di sodium Lauryl Sulfosuccinate, Disodium Laureth Sulfosuccinate, and Disodium Cocoyl Sulfosuccinate, for example. In particular, Disodium Laureth Sulfosuccinate is preferred. Corresponding commercial products are available, for example, from Evonik under the trade name “Rewopol®” or from BASF under the trade name “Texapon SB®.”

Preferred “C₂ to C₄ monoalkanolamides and/or dialkanolamides of at least one C₈ to C₂₄ carboxylic acid” shall preferably be understood to mean the ethanolamides and/or isopropanolamides of C₈ to C₂₄ carboxylic acids, wherein the representatives derived from coconut fatty acids, lauric acid, myristic acid, palmitic acid and stearic acid are particularly preferred. Particularly preferred C₂ to C₄ monoalkanolamides and/or dialkanolamides according to the invention are those known by the INCI nomenclature Cocamide MEA, Cocamide DEA, Cocamide MIPA, Lauramide MEA, Lauramide DEA, Lauramide MIPA, Palmitoylamid MEA, Pahnitoylamid DEA, Stearamid MEA, and Stearamid DEA. Within the scope of the present invention, in particular the compounds known by the INCI names Cocamide MEA, Cocamide MIPA and/or Lauramide MEA are preferred.

In a first preferred embodiment, cosmetic cleansing agents according to the invention are characterized by comprising
a) at least one anionic surfactant according to formula (I), in which
   at least one of the groups R² to R⁵ denotes a methyl group, and the remaining groups denote a hydrogen atom;
   R¹ denotes a linear or branched, saturated or unsaturated alkyl group having 8 to 18 carbon atoms; and
M⁺ denotes a sodium, potassium or ammonium ion;
b) at east one anionic surfactant according to formula (II), in which
   R′ denotes a linear, saturated alkyl group having 8 to 18 carbon atoms;
   n denotes a number from 1 to 8; and
   X denotes a sodium, potassium or ammonium ion; and
c) at least one monoethanolamide of a C₈ to C₁₈ carboxylic acid.

Within this embodiment, particularly preferred cosmetic cleansing agents are those that comprise
a) at least one of the anionic surfactants known by the INCI names Sodium Lauroyl Methyl Isethionate or Sodium Cocoyl Methyl Isethionate;
b) at least one anionic surfactant known by the INCI name Disodium Laureth Sulfosuccinate; and
c) at least one compound known by the INCI name Cocamide MEA, Cocamide MIPA or Lauramide MEA.

So as to generate an optimal amount of foam, it is preferred according to the invention if surfactants a), h) and c) are present in the cosmetic cleansing agents according to the invention in certain quantity ranges:
anionic surfactant a): 1 to 20 wt. %;
anionic surfactant b): 0.5 to 15 wt. %; and
nonionic surfactant c): 0.1 to 5 wt. %;
   wherein the quantity information is based on the total weight of the cleansing agent.

In a second preferred embodiment, cosmetic cleansing agents according to the invention are thus characterized in that
the percentage by weight of the at least one anionic surfactant according to formula (I) based the total weight of the composition is preferably 1 to 20 wt, more preferably 2 to 15 wt. %, and in particular at least 3 to 10 wt. %;
the percentage by weight of the at least one anionic surfactant according to formula (II) based the total weight of the composition is preferably 0.5 to 15 wt. %, more preferably 1 to 10 wt. %, and in particular at least 2 to 7.5 wt. %; and
the percentage by weight of the at least one C₂ to C₄ monoalkanolamide and/or dialkanolamide of at least one C₈ to C₂₄ carboxylic acid based the total weight of the composition is preferably 0.1 to 5 wt. %, more preferably 0.5 to 4 wt. %, and in particular 1 to 3 wt. %.

It was found that the feel of the foam generated by way of the agents according to the invention and the creaminess of the foam can be enhanced even further if the cleansing agents contain the surfactants a), b) and c) in a certain weight ratio.

In a third preferred embodiment, cosmetic cleansing agents according to the invention are thus characterized by preferably comprising the surfactants a), b) and c) in a weight ratio of (1-4):(1-3):(0.5-2), and particularly preferably of (2-4):(1.5-2.5):(0.75-1.5).

It was a goal of the invention to provide particularly mild, strongly foaming cosmetic compositions, which was able to be achieved by the combination of surfactants a), b) and c). The amount of foam generated by the agents according to the invention approximately corresponds to the amount of foam that can be achieved with alkyl ether sulfate-based cleansing agents, wherein the agents according to the invention are milder. The additional use of anionic alkyl ether sulfates in the cleansing agents according to the invention is therefore not necessary.

In a further preferred embodiment of the invention, cosmetic cleansing agents according to the invention are therefore characterized in that the percentage by weight of sulfate group-containing surfactant based on the total weight of the compositions is preferably less than 0.5 wt. %, more preferably less than 0.3 wt. %, and in particular less than 0.2 wt. %.

In contrast, it was possible to increase the amount of foam, the foam properties (in particular the high foam density) and/or the mildness of the compositions according to the invention even further when specific amphoteric surfactants were added. Suitable amphoteric and/or zwitterionic surfactants preferably correspond to at least one compound of the following formulas (i) to (vii), in which the group R denotes a straight-chain or branched, saturated or monounsaturated or polyunsaturated alkyl or alkenyl group having 7 to 23 carbon atoms (formulas (i) and (ii)) or a straight-chain or branched, saturated or monounsaturated or polyunsaturated alkyl or alkenyl group having 8 to 24 carbon atoms (formulas (iii) to (vii)).

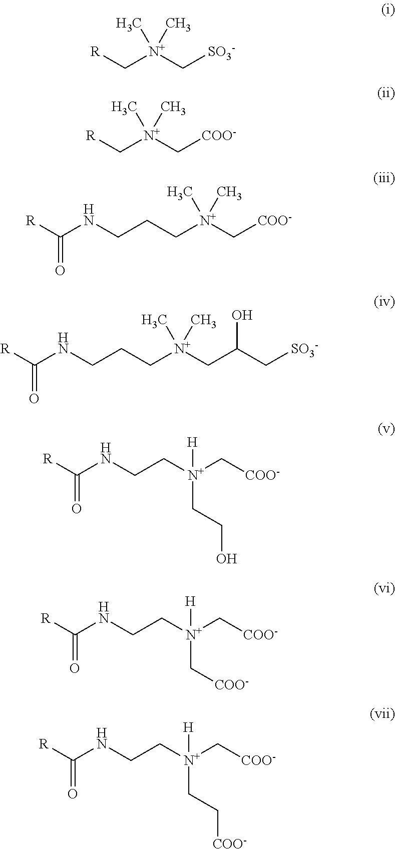

Preferred amphoteric and/or zwitterionic surfactants of one of the aforementioned formulas (i) to (vii) predominantly contain a straight-chain or branched, saturated or monounsaturated or polyunsaturated alkyl group having 8 to 20, more preferably 8 to 16, and in particular 8 to 12 carbon atoms as the group R. Amphoteric and/or zwitterionic surfactants in which the group R is derived from coconut oil are more preferred. Amphoteric and/or zwitterionic surfactants of formulas (iii), (v), (vi) and (vii) are particularly preferred.

In particular, the amphoteric surfactants known by the INCI names Cocamidopropyl Betaine and/or Cocoampho(di)acetate, which are commercially available from several suppliers, are preferred. The percentage by weight of the amphoteric/zwitterionic surfactant(s) of one of the aforementioned formulas (i) to (vii) based on the total weight of the cosmetic agents of the use according to the invention is preferably 0.1 to 15 wt. %, preferably 0.5 to 12.5 wt. %, and in particular 1 to 10 wt. %,

In a further preferred embodiment of the invention, cosmetic cleansing agents according to the invention are therefore characterized by additionally comprising at least one amphoteric surfactant, preferably an amphoteric surfactant according to one of the formulas (i) to (vii), wherein the percentage by weight of the amphoteric surfactant based on the total weight of the composition is preferably 0.1 to 15 wt., more preferably 0.5 to 12.5 wt., and in particular 1 to 10 wt.

So as to further optimize the hair care properties of the cosmetic cleansing agents according to the invention, it is advantageous when these additionally contain a conditioning active ingredient complex d), in addition to the surfactant mixture a), b) and c). It was found that specific active ingredient complexes in combination with the surfactant combination a), b) and c) are particularly suitable for imparting a high care effect to the cleansing agents according to the invention. In particular, the wet and dry combability of the hair was able to be increased even further by adding the active ingredient complex.

In a fourth preferred embodiment, cosmetic cleansing agents according to the invention are thus characterized by additionally comprising at least one conditioning active ingredient complex d), containing at least one silicone compound and at least one cationic polymer.

Silicones effectuate outstanding conditioning properties on the hair. In particular, they positively affect the feel of the hair and the softness of the hair in many instances. It is therefore desirable to use silicones in cosmetic hair treatment products. Suitable silicone compounds within the meaning of the present invention can be selected from:
(i) polyalkylsiloxanes, polyarylsiloxanes, polyalkylarylsiloxanes, which are volatile or non-volatile, straight-chain, branched or cyclic, cross-linked or not cross-linked;
(ii) polysiloxanes containing, in the general structure thereof, one or more organofunctional groups, which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane(A)/polyoxyalkylene(B) block copolymers of the (A-B)ₙ type where n>3;
(iv) grafted silicone polymers having a non-silicone-containing, organic skeleton, which are composed of an organic main chain formed of organic monomers that do not contain any silicone, onto which at least one polysiloxane macromer was grafted in the chain and optionally on at least one chain end;
(v) grafted silicone polymers having a polysiloxane skeleton onto which non-silicone-containing, organic monomers were grafted that include a polysiloxane main chain, onto which at least one organic macromer that does not contain silicone was grafted in the chain and optionally on at least one of the ends thereof
(vi) or the mixtures thereof

Examples of silicone compounds that are particularly suited according to the invention are:
a) silicones of the following formula (III)

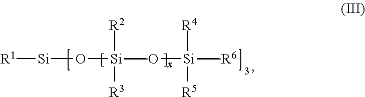

in which
R¹ to R⁵ independently of one another denote an alkyl, aryl or alkyl aryl group having 1 to 30 carbon atoms;
R⁶ denotes a group carrying at least one quaternary ammonium group;
x denotes a number in the range from 2 to 200; and/or
b) silicones of the following formula (IV)

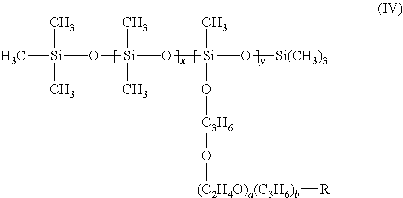

in which
the group R denotes a hydrogen atom, an alkyl group having 1 to 12 carbon atoms, an alkoxy group having 1 to 12 carbon atoms or a hydroxyl group;
x denotes a number from 1 to 100, preferably from 20 to 30;
y denotes a number from 1 to 20, preferably from 2 to 10; and
a and b denote numbers from 0 to 50, preferably from 10 to 30.

In preferred silicones according to the invention according to formula (III),
x denotes a number from 3 to 120;
the group R¹ denotes an aryl group, and preferably a phenyl group;
the groups R² to R⁵ denote identical C₁ to C₄ alkyl groups, and preferably methyl groups;
the group R⁶ denotes a group having the composition R⁷-R⁸, wherein
the grouping R⁷ denotes at least one grouping of the following formulas (V) to (VIII)

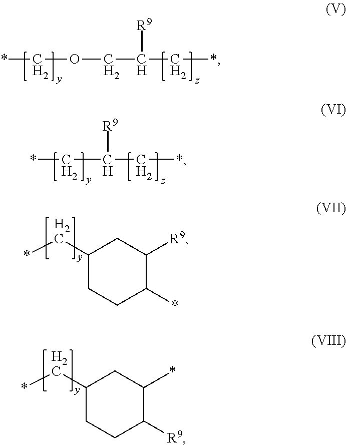

in which
y denotes a number from 2 to 18;
z denotes a number from 0 to 3; and
R⁹ denotes hydrogen, a C₁ to C₄ alkyl group, a C₂ to C₄ hydroxyalkyl group or an OH group; and
the group R⁸ corresponds to at least one of the follo ring formulas (IX) or (X)

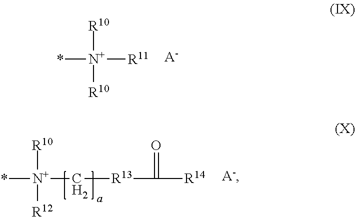

in which
R¹⁰ and R¹² each independently of one another denote hydrogen or an alkyl group having 1 to 30 carbon atoms;
R¹¹ denotes an alkyl group having 1 to 30 carbon atoms or a grouping (CH)₂)_{b}—NR¹⁰R¹², in which b denotes a number from 2 to 18;
R¹³ denotes a grouping —O— or —N(H)—;
R¹⁴ denotes an alkyl group having 1 to 30 carbon atoms;
a denotes a number from 2 to 18; and
A⁻ denotes inorganic or organic acid anions.

Suitable C₁ to C₄ alkyl groups shall be understood to mean methyl, ethyl, n-propyl, isopropyl and n-butyl groups. Methyl and ethyl groups are preferred. In particular, methyl groups are preferred.

Particularly preferred are silicone compounds according to formula in which
x denotes a number from 3 to 120;
the group R¹ is a phenyl group;
the groups R² to R⁵ are methyl groups;
the group R⁶ denotes a group having the composition R⁷-R⁸, wherein
the grouping R⁷ corresponds to a grouping of formula (V); and
the group R⁸ corresponds to formula (X).

In particular, silicone compounds according to formula (III) are preferred in which
x denotes a number from 8 to 80;
the group R¹ is a phenyl group;
the groups R² to R⁵ are methyl groups;
the group R⁶ denotes a group having the composition R⁷-R⁸, wherein
the grouping R⁷ denotes a grouping of formula (V) in which y is the number 3, z is the number 1, and R⁹ denotes an OH group; and
the group R⁸ corresponds to formula (X) in which R¹⁰ and R¹² denote methyl groups, a denotes the number 3, R¹³ denotes the grouping —N(H)—, and R¹⁴ denotes a C₈ to C₁₈ alkyl group.

A particularly suitable silicone compound according to formula (III) for the cosmetic cleansing agents according to the invention is known, for example, by the INCI name Silicone Quaternium-22 and is commercially available. One example of a commercial product that is based on a silicone compound by the INCI name Silicone Quaternium-22 and can be used in the cleansing agents according to the invention is Abil® T Quat 60 from Evonik. Abil® T Quat 60 contains a mixture of the active ingredients known by the INCI names Silicone Quaternium-22 (65%) and PPG-3 Myristyl Ether (35%).

Preferred silicones according to formula (IV) are water-soluble. Within the meaning of the invention, “water-soluble” shall be understood to mean silicones that have a solubility of at least 2 g/100 g water under standard conditions (25° C., 1013 mbar). Particularly preferred silicones b) have at least a water solubility, under standard conditions in each case, of at least 5 g/100 g water, more preferably of at least 10 g/100 g water, and in particular preferred silicones according to formula (IV) are completely water-soluble, which shall be understood to mean that they form a solution at any ratio with water. Further preferred silicones according to formula (IV) are non-volatile.

It has been found that it is advantageous for easier production of the cosmetic cleansing agents according to the invention if the silicones b) are liquid under standard conditions (25° C., 1013 mbar). Particularly preferred silicones according to formula (IV) are known, for example, by the CTFA name Dimethicone Copolyol and are commercially available under the designations Silsoft® (Momentive), Silwet® (General Electric), Dow Corning® (Dow), Belsil® (Wacker), and Abil® (Goldschmidt), for example. Preferred commercial products that contain silicones according to formula (IV) by the CTFA name Dimethicone Copolyol and can be used in the cleansing agents according to the invention are, for example, Silsoft® 895 (Momentive), Silwet© L 722 (General Electric), Silwet® 7001 (General Electric), Silwet® 7605 (General Electric), Silwet® 7500 (General Electric), Dow Corning® 190 (Dow), Dow Corning® 193 (Dow), Dow Corning® 3225 (Dow), Belsil® DMC 6031 (Wacker) and/or Abil® 8842 (Goldschmidt). The percentage of weight of the at least one silicone compound based on the total weight of the cosmetic cleansing agents is preferably 0.01 to 10 wt. %, more preferably 0.05 to 7.5 wt. %, particularly preferably 0.075 to 6 wt. %, and in particular 0.1 to 4 wt. %.

Suitable cationic polymers are, for example:
quaternized cellulose derivatives, as they are commercially available under the designations Ceiquat® and Polymer JR®;
quaternized cellulose derivatives, which may be hydrophobically modified, for example Polyquaternium-67,
cationic alkyl polyglycosides;
cationized honey, for example the commercial product Honeyquat® 50;
cationic guar derivatives, such as in particular the products sold under the trade names Cosmedia®Guar N-Hance® and Jaguar®;
polymeric dimethyl diallyl ammonium salts and the copolymers thereof with esters and amides of acrylic acid and methacrylic acid. The products available commercially under the names Merquat® 100 (poly(dimethyldiallylammonium chloride)) and Merquat® 550 (dimethyldiallylammonium chloride/acrylamide copolymer) are examples of such cationic polymers;
copolymers of vinylpyrrolidone with quaternized derivatives of dialkyl aminoalkyl acrylate and methacrylate, such as diethyl sulfate-quaternized vinylpyrrolidone/dimethylaminoethyl methacrylate copolymers. Such compounds are commercially available under the designations Gafquat®734 and Gafquat® 755;
vinylpyrrolidone/vinylimidazolium methochloride copolymers, as they are offered under the names Luviquat® FC 370, FC 550, FC 905 and HM 552;
quaternized polyvinyl alcohol;
and the polymers known by the designations
Polyquaternium 2, Polyquaternium 17, Polyquaternium 18, Polyquaternium-24, Polyquaternium 27, Polyquaternium-32, Polyquatemium-37, Polyquaternium 74, and Polyquaternium 89.

Preferred cationic polymers are preferably selected from the polymers known by the INCI names Guar Hydroxypropyltrimonium Chloride, Polyquaternium-67, Polyquaternium 10, Polyquaterniu-6, Polyquatemium-7 and/or Polyquaternium-37. In particular, the polymers known by the INCI names Guar Hydroxypropyltrimonium Chloride, Polyquaternium-6, Polyquaternium-7, and Polyquaternium-10 are preferred. The percentage of weight of the cationic polymer or polymers based on the total weight of the cosmetic cleansing agents is preferably 0.01 to 10 wt. %, more preferably 0.025 to 7.5 wt. %, particularly preferably 0.05 to 6 wt. %, and in particular 0.1 to 5 wt. %.

The fourth preferred embodiment particularly preferably comprises cosmetic cleansing agents according to the invention that contain at least one conditioning active ingredient complex d) which includes
a) a silicone compound of the following formula

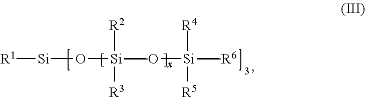

in which
R¹ to R⁵ independently of one another denote an alkyl, aryl or alkyl aryl group having 1 to 30 carbon atoms;
R⁶ denotes a group carrying at least one quaternary ammonium group;
x denotes a number in the range from 2 to 200; and/or
b) a silicone compound of the following formula (IV)

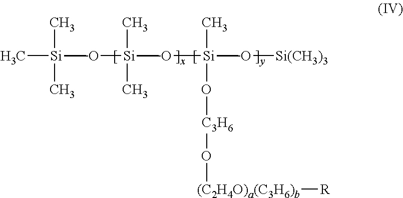

in which
the group R denotes a hydrogen atom, an alkyl group having 1 to 12 carbon atoms, an alkoxy group having 1 to 12 carbon atoms or a hydroxyl group;
x denotes a number from 1 to 100, preferably from 20 to 30;
y denotes a number from 1 to 20, preferably from 2 to 10; and
a and b denote numbers from 0 to 50, preferably from 10 to 30; and
c) at least one cationic polymer, selected from quaternized cellulose polymers, cationic guar derivatives and/or cationic polymers based on acrylic acid (derivatives).

In particular, preferred cosmetic cleansing agents within this fourth preferred embodiment preferably comprise at least one silicone compound known by the INCI name Silicone Quaternium-22 and/or at least one silicone compound known by the INCI name PEG-12 Dimethicone, and at least one cationic polymer known by the INCI names Polyquatemium-6, Polyquaternium-7, Polyquaternium-10 and/or Guar Hydroxypropyltrimonium Chloride as the active ingredient complex d). The percentage by weight of the conditioning active ingredient complex d) based the total weight of the composition is preferably 0.1 to 10 wt. %, more preferably 0.25 to 7.5 wt. %, and in particular 0.5 to 5 wt. %.

In a fifth preferred embodiment of the invention, cosmetic cleansing agents according to the invention are characterized by comprising
a) at least one anionic surfactant according to formula (I), in which
   at least one of the groups R² to R⁵ denotes a methyl group, and the remaining groups denote a hydrogen atom;
   R¹ denotes a linear or branched, saturated or unsaturated alkyl group having 8 to 18 carbon atoms; and
   M⁺ denotes a sodium, potassium or ammonium ion;
b) at least one anionic surfactant according to formula (II), in which
   R′ denotes a linear, saturated alkyl group having 8 to 18 carbon atoms;
   n denotes a number from 1 to 8; and
   X denotes a sodium, potassium or ammonium ion; and
c) at least one monoethanolamide of a C₈ to C₁₈ carboxylic acid; and
d) at least one active ingredient complex d), which contains at least one silicone compound and at least one cationic polymer, wherein
   the percentage of weight of the at least one surfactant a) based on the total weight of the cosmetic cleansing agents is preferably 1 to 20 wt. %;
   the percentage of weight of the at least one surfactant b) based on the total weight of the cosmetic cleansing agents is preferably 0.5 to 15 wt. %;
   the percentage of weight of the at least one surfactant c) based on the total weight of the cosmetic cleansing agents is preferably 0.1 to 5 wt. %; and
   the percentage of weight of the active ingredient complex d) based on the total weight of the cosmetic cleansing agents is preferably 0.1 to 10 wt. %.

Within this embodiment, particularly preferred cleansing agents according to the invention are those that comprise
a) at least one of the anionic surfactants known by the INCI names Sodium Lauroyl Methyl Isethionate or Sodium Cocoyl Methyl Isethionate;
b) at least one anionic surfactant known by the INCI name Disodium Laureth Sulfosuccinate;
c) at least one compound known by the INCI name Cocamide MEA, Cocamide MIPA or Lauramide MEA; and
d) at least one active ingredient complex d), which contains at least one silicone compound known by the INCI name Silicone Quaternium-22 and/or at least one silicone compound known by the INCI name PEG-12 Dimethicone, and at least one cationic polymer known by the INCI names Polyquaternium-6, Polyquatemium-7, Polyquaternium-10 and/or Guar Hydroxypropyltrimonium Chloride, wherein
   the percentage of weight of the at least one surfactant a) based on the total weight of the cosmetic cleansing agents is preferably 3 to 10 wt. %;
   the percentage of weight of the at least one surfactant b) based on the total weight of the cosmetic cleansing agents is preferably 2 to 7.5 wt. %;
   the percentage of weight of the at least one surfactant c) based on the total weight of the cosmetic cleansing agents is preferably 1 to 3 wt. %; and
   the percentage of weight of the active ingredient complex d) based on the total weight of the cosmetic cleansing agents is preferably 0.5 to 5 wt. %.

In a further preferred embodiment, the care properties of the cosmetic agents may be further enhanced if they additionally include at least one further conditioning active ingredient, which may be selected from the group of
protein hydrolysates;
vitamins;
fats, oils and/or waxes;
glycerol.

Suitable protein hydrolysates shall be understood to mean product mixtures that can be obtained by the acidically, basically or enzymatically catalyzed degradation of proteins. Protein hydrolysates of plant, animal and/or marine origin can be used. Animal protein hydrolysates are, for example, elastin, collagen, keratin, silk and milk protein hydrolysates, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda). Protein hydrolysates of plant origin, such as soy, almond, rice, pea, potato and wheat protein hydrolysates, are preferred. Such products are vailable, for example, m ple, under the tradearks Gluadin® (Cognis), DiaMin® a Diamalt), Lexein® (Inox), and Crotein® (Croda).

It is also possible to use cationized protein hydrolysates, wherein the underlying protein hydrolysate can stem from animals, for example from collagen, milk or keratin, from plants, for example from wheat, maize, rice, potatoes, soy or almonds, from marine life forms, for example from fish collagen or algae, or from protein hydrolysates obtained by way of biotechnology. The protein hydrolysates underlying the cationic derivatives can be obtained from the corresponding proteins by way of a chemical, in particular alkaline or acid hydrolysis, an enzymatic hydrolysis and/or a combination of the two types of hydrolysis. The hydrolysis of proteins generally results in a protein hydrolysate having a molecular weight distribution from approximately 100 daltons to several thousand daltons Cationic protein hydrolysates in which the underlying protein component has a molecular weight of 100 to 25000 daltons, preferably 250 to 5000 daltons, are preferred.

Moreover, cationic protein hydrolysates shall be understood to mean quaternized amino acids and the mixtures thereof The quaternization of the protein hydrolysates or of the amino acids is frequently carried out using quaternary ammonium salts, such as N,N-dimethyl-N-(n-alkyl)-N-(2-hydroxy-3-chloro-n-propyl) ammonium halides. The cationic protein hydrolysates can moreover also be further derivatized. Typical examples of the cationic protein hydrolysates and derivatives that shall be mentioned are the commercially available products known under the following NCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The percentage by weight of the protein hydrolysate or protein hydrolysates based the total weight of the cosmetic agent is preferably 0.1 to 5 wt. %, more preferably 0.025 to 3 wt. %, and in particular 0.05 to 2 wt. %.

Suitable vitamins shall preferably be understood to mean the following vitamins, provitamins, and vitamin precursors, and the derivatives thereof:
Vitamin A: The group of substances referred to as vitamin A includes retinol (vitamin A₁) and 3,4-didehydroretinol (vitamin A₂). β-carotene is the provitamin of retinol. Possible vitamin A components are, for example, vitamin A acid and the esters thereof, vitamin A aldehyde, and vitamin A alcohol and the esters thereof, such as palmitate and acetate.
Vitamin B: The vitamin B group or vitamin B complex includes, among other things.
   Vitamin B₁ (thiamine)
   Vitamin B₂ (riboflavin)
   Vitamin B₃. The compounds nicotinic acid and nicotinic acid amide (niacinamide) are often included under this designation.
   Vitamin B₅ (pantothenic acid and panthenol). Within this group, panthenol is preferably used. Derivatives of panthenol that can be used are in particular the esters and ethers of panthenol, pantolactone and cationically derivatized panthenols. Individual representatives are, for example, panthenol triacetate, panthenol monoethyl ether and the monoacetate thereof, and cationic panthenol derivatives.
   Vitamin B₆ (pyridoxine, pyridoxamine and pyridoxal).
Vitamin C (ascorbic acid): Use in the form of the palmitic acid ester, glucosides or phosphates can be preferred. Use in combination with tocopherols can likewise be preferred.
Vitamin E (tocopherols, in particular α-tocopherol).
Vitamin F: The term “vitamin F” is usually understood to mean essential fatty acids, in particular linoleic acid, linolenic acid and arachidonic acid. •Vitamin H:
Vitamin H refers to the compound (3aS,4S,6aR)-2-oxohexahydrothienol[3,4-d]-imidazole-4-valeric acid, although this has since become established by the trivial name biotin.

Vitamins, provitamins and vitamin precursors from the groups A, B, E and H are particularly preferred. In particular, nicotinic acid amide, biotin, pantolactone and/or panthenol are preferred. The percentage by weight of the vitamin(s), vitamin derivative(s) and/or vitamin precursor(s) based the total weight of the cosmetic agents is preferably 0.001 to 2 wt. %, particularly preferably 0.005 to 1 wt. %, and in particular 0.01 to 0.5 wt. %.

Suitable oil, wax and/or fat components can preferably be selected from mineral or natural oil components and/or fats. Usually, triglycerides and mixtures of triglycerides are used as natural (vegetable) oils. Preferred natural oils are coconut oil, (sweet) almond oil, walnut oil, peach kernel oil, apricot kernel oil, avocado oil, tea tree oil, soy bean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter and/or shea butter.

Mineral oils that are used are in particular petroleum, paraffin and isoparaffin oils, and synthetic hydrocarbons. One example of a hydrocarbon that can be used, for example, is 1,3-di-(2-ethylhexyl)cyclohexane (Cetiol® S), which is available as a commercial product. A dialkyl ether can furthermore serve as the oil component.

Suitable dialkyl ethers are in particular di-n-alkyl ethers having in total between 12 and 36 carbon atoms, in particular 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, and di-tert-butyl ether, di-iso-pentyl ether, di-3-ethyldecyl ether, tert-butyl-n-octyl ether, iso-pentyl-n-octyl ether and 2-methyl-pentyl-n-octyl ether. The di-n-octyl ether that is commercially available under the designation Cetiol® OE is particularly preferred.

Fats shall be understood to mean fatty acids, fatty alcohols and natural and synthetic waxes, which may be present either in solid form or liquid in an aqueous dispersion. Linear and/or branched, saturated and/or unsaturated fatty acids having 6 to 30 carbon atoms can be used as fats. Fatty acids having 10 to 22 carbon atoms are preferred. Among these, for example, isostearic acids should be mentioned, such as the commercial products Emersol® 871 and Emersol® 875, and isopalmitic acids, such as the commercial product Edenor® IP 95, and all further fatty acids sold under the trade name Edenor® (Cognis). Further typical examples of such fatty acids are caproic acid, caprylic acid, 2-ethyl-hexanic acid, caprinic acid, lauric acid, isotridecanic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and the technical mixtures thereof.

Usually, the fatty acid cuts that are obtainable from coconut oil or palm oil are particularly preferred; in particular, the use of stearic acid is generally preferred.

Suitable fatty alcohols are saturated, monounsaturated or polyunsaturated, branched or unbranched fatty alcohols having C₆ to C₃₀, preferably C₁₀ to C₂₂, and most particularly preferably C₁₂ to C₂₂ carbon atoms. It is possible to use, for example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, and the guerbet alcohols thereof, wherein this enumeration has be of an exemplary and non-limiting nature. The fatty alcohols, however, are preferably derived from natural fatty acids, wherein usually a production from the esters of fatty acids by way of reduction can be assumed. According to the invention, it is likewise possible to use fatty alcohol cuts that are produced by reducing naturally occurring triglycerides, such as suet, palm oil, peanut oil, rapeseed oil, cottonseed oil, soy bean oil, sunflower oil, and linseed oil, or from the transesterification products thereof with appropriate alcohols, and thus represent a mixture of different fatty alcohols. Such substances are available for purchase, for example, under the designations Stenol®, such Stenol® 1618, or Lanette®, such as Lanette® O, or Lorol®, such as Lorol® C8, Lorol® C14, Lorol® C18, Lorol® C8-18, HD-Ocenol®, Crodacol®, such as Crodacol® CS, Novol®, Eutanol® G, Guerbitol® 16, Guerbitol® 18, Guerbitol® 20, Isofol® 12, Isofol® 16, Isofol® 24, Isofo® 36, isocarb® 12, Isocarb® 16 or Isocarb® 24. It is also possible according to the invention, of course, to use wool wax alcohols, as they are available for purchase, for example, under the designations Corona®, White Swan®. Coronet® or Fluilan®. Suitable natural or synthetic waxes that can be used are solid paraffins or isoparaffins, carnauba waxes, beeswaxes, candelilla waxes, ozokerites, ceresin, spermaceti wax, sunflower wax, fruit waxes such as apple wax or citrus wax, and microwaxes made of PE or PP. Such waxes are available from Kahl & Co., Trittau, for example,

Further fats are, for example
ester oils. Ester oils shall be understood to mean the esters of C₆ to C₃₀ fatty acids having C₂ to C₃₀ fatty alcohols. The monoesters of fatty acids with alcohols having 2 to 24 carbon atoms are preferred. Examples of fatty acid components used in the esters are caproic acid, caprylic acid, 2-ethyl-hexanic acid, caprinic acid, lauric acid, isotridecanic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and the technical mixtures thereof Examples of fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, eleostearyl alcohol, arachyl alcohol, gadoleyl alcohol, behenyl alcohol, erucyl alcohol, and brassidyl alcohol, and the technical mixtures thereof Particularly preferred are isopropyl myristate (Rilanit® IPM), isononanoic acid-C₁₆₋₁₈ alkyl ester (Cetiol® SN), 2-ethylhexyl palmitate (Cegesoft® 24), stearic acid-2-ethylhexyl ester (Cetiol® 868), cetyl oleate, glycerin tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelaate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate;
symmetrical, asymmetrical or cyclic esters of carbonic acid comprising fatty alcohols, glycerol carbonate or di-n-caprylyl carbonate (Cetiol® CC);
ethoxylated and/or non-ethoxylated mono-, di- and tri-fatty acid esters of saturated and/or unsaturated linear and/or branched fatty acids with glycerol, such as Monomuls® 90-O18, Monomuls® 90-L12, Cetiol® HE or Cutina® MD.

The percentage by weight of the oil, wax and/or fat components based the total weight of the cosmetic agents is preferably 0.01 to 5 wt. %, particularly preferably 0.025 to 4 wt. %, and in particular 0.05 to 3 wt. %. Glycerol can be added separately to the cosmetic agents in an amount of up to 10 wt. % (based on the total weight of the cleansing agent). However, it can also be a component of the aqueous-alcoholic carrier.

It was found that the cosmetic agents according to the invention are also suitable for use as an anti-dandruff preparation. The total weight of anti-dandruff agents based on the total weight of the cosmetic agents can preferably be 0.01. to 10 wt. %, more preferably 0.025 to 7.5 wt. %, particularly preferably 0.05 to 5 wt. %, and in particular 0.075 to 3 wt. %. Suitable active anti-dandruff ingredients can be selected from piroctone olamine, climbazole, zinc pyrithione, ketoconazoles, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts. Climbazole, zinc pyrithione and piroctone olamine are preferred.

Further active ingredients, auxiliary substances and additives that can be present in the cosmetic agents according to the invention are, for example:
plant extracts;
humectants;
perfumes;
UV filters;
thickening agents such as gelatin or gums, for example agar-agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays and phyllosilicates, such as bentonite or fully synthetic hydrocolloids, such as polyvinyl alcohol, the Ca, Mg or Zn soaps;
thickening agents such as acryl and methacryl (co)polymers, for example the cross-linked homopolymers of acrylic acid (INCI name: Carbomer), which are also referred to as carboxyvinyl polymers. Such polyacrylic acids are available, among other things from 3V Sigma under the trade name Poygel®, and from B.F. Goodrich under the trade name Carbopol®, such as Carbopol 940 (molecular weight approximately 4,000.000), Carbopol 941 (molecular weight approximately 1,250,000) or Carbopol 934 (molecular weight approximately 3,000,000). Furthermore, for example, the following acrylic acid copolymers are suited:
a. copolymers of two or more monomers from the group consisting of acrylic acid, methacrylic acid, and the simple esters thereof, preferably formed with C₁ to C₄ alkanols (INCI name: Acrylates Copolymer), which include, for example, the copolymers of methacrylic acid, butyl acrylate and methyl methacrylate or of butyl acrylate and methyl methacrylate, and which are available, for example, from Rohm & Haas under the trade name Aculyn® and Acusol® and from Degussa (Goldschrnidt) under the trade name Tego® Polymer, such as Aculyn 22, Aculyn 28, Aculyn 33 (cross-linked), Acusol 810, Acusol 820, Acusol 823, and Acusol 830;
b. cross-linked high molecular weight acrylic acid copolymers, which include, for example, the copolymers, cross-linked with an allyl ether of sucrose or pentaerythrite, of C₁₀ to C₃₀ alkyl acrylates with one or more monomers from the group consisting of acrylic acid, methacrylic acid, and the simple esters thereof, preferably formed with C₁ to C₄ alkanols, (INCI name: Acrylates-(C₁₀-C₃₀)-Alkyl Acrylate Crosspolymer), and which are available, for example, from B.F. Goodrich under the trade name Carbopol®, such as Carbopol ETD 2020 and Carbopol 1382 (INCI Acrylates-(C₁₀-C₃₀)-Alkyl Acrylate Crosspolymer) and Carbopol Aqua 30;
structurants such as maleic acid and lactic acid;
dyes for coloring the agent;
substances for setting the pH value, for example α- and β-hydroxycarboxylic acids such as citric acid, lactic acid, malic acid, glycolic acid;
active ingredients such as bisabolol;
complexing agents such as EDTA, NTA, β-alanine diacetic acid, and phosphonic acids;
ceramides. Ceramides shall be understood to mean N-acylsphingosine (fatty acid amides of sphingosine) or synthetic analogs of such lipids (known as pseudoceramides);
blowing agents such as propane-butane mixtures, N₂O, dimethylether, CO₂, and air;
antioxidants;
preservatives, such as sodium benzoate or salicylic acid;
additional viscosity regulators such as salts (NaCl).

The cosmetic agents according to the invention can preferably have a pH value in the range of 3 to 7, more preferably of 4 to 6.5, particularly preferably of 4.5 to 6.6, and in particular of 5 to 6.

The cosmetic agents according to the invention preferably have a viscosity in the range of 1000 to 15000 mPas, preferably 1500 to 12500 mPas, and in particular 3000 to 10000 mPas (in each case measured using a Haake rotational viscometer VT550; 20° C.; measuring device MV; spindle MV II; 8 rpm). Compositions having such viscosity are easy to distribute on the respective application surface, particularly preferably on the hair, and can optionally be rinsed off again with water after the application.

### EXAMPLES

The following cleansing compositions according to the invention were produced (compositions A and B of the table below; quantity information in [wt. %]):

| | A | B |
|---|---|---|
| Tego Betain F50 ®¹ | 10.0 | 9.0 |
| Iselux LQ ®² | 20.0 | 18.0 |
| Texapon SB 3 ®³ | 12.0 | 14.0 |
| Rewoteric AM C ®⁴ | 6.0 | 8.0 |
| Comperlan 100 ®⁵ | 2.5 | 3.0 |
| Abil T Quat 65 ®⁶ | 0.5 | 1.0 |
| JR 400 ®⁶ | 0.7 | |
| Merquat 550 ®⁷ | 4.0 | 5.0 |
| Xiameter OFX-0193 ®⁸ | 0.5 | 0.7 |
| PEG-40 Hydrogenated Castor Oil | 0.3 | 0.6 |
| TiO₂ | 0.05 | |
| Carbopol ETD 2020 ®⁹ | 0.6 | 0.5 |
| Preservative | q.s. | q.s. |
| Perfume | q.s. | q.s. |
| Citric acid/NaOH (pH 5.5-6) | q.s. | q.s. |
| Water | ad 100 | ad 100 |

| | |
|---|---|
| Appearance | white, milky |
| Viscosity* | 4000-8000 mPas |

| | |
|---|---|
| Determination of the foam quality | 1-2 |
| Reduction of wet combability [%] | 76 |
| Determination of combability in dry hair | 2 |

*determined by way of a Haake rotational viscometer VT500; 20° C.; measuring device MV; spindle MV II; 8 rpm

The following commercial products were used in compositions A and B:
¹INCI name: AQUA, COCAMIDOPROPYL BETAINE; 35-37, 5% AS; Evonik
²INCI name: SODIUM LAUROYL METHYL ISETHIONATE; 32% AS; Innospec
³INCI name: AQUA (WATER), DISODIUM LAURETH SULFOSUCCINATE, CITRIC ACID; approximately 40% AS; BASF
⁴INCI name: SODIUM COCOAMPHOACETATE; 50% AS; Evonik
⁵INCI name: COC AMIDE MEA; BASF
⁶INCI name: SILICONE QE ATERNIUM-22; Evonik
⁷INCI name: POLYQU ATERN I U M-7; 8.5-0.9% AS; Lubrizol
⁸INCI name: PEG-12 DIMETHICONE; Dow Corning
⁹INCI name: ACRYLATES/C 10-30 ALKYL ACRYLATE CROSSPOLYMER; Lubrizol.

The esults in the table show that the foam quality of composition A according to the invention was found by experts to be good on a scale of 1 to 6 (1=very good, 6=poor). The evaluation with respect to the quality of the foam included the amount of foam, the feel, and the creaminess of the foam.

The compositions according to the invention furthermore allowed the care of the hair during cleansing to be improved, which was noticeable in particular in the improved combability of the hair (wet and dry combability).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleansing agent, including in at least one suitable carrier
a) at least one first anionic surfactant according to the following formula (I) 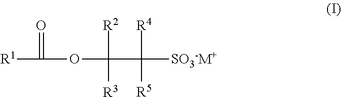
in which
at least one of the groups R² to R⁵ denotes a C₁ to C₄ alkyl group, and the remaining groups independently of one another denote a hydrogen atom or a C₁ to C₄ alkyl group;
R¹ in each case denotes a linear or branched, saturated or unsaturated alkyl group having 6 to 30 carbon atoms; and
M⁺ in each case denotes an ammonium, alkanol ammonium or metal cation;
b) at least one second anionic surfactant of the following formula (II) 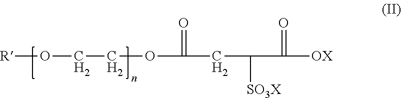
in which
R′ denotes a linear or branched, saturated or unsaturated alkyl group having 6 to 30 carbon atoms;
n denotes a number from 0 to 20, preferably from 1 to 10; and
X denotes an ammonium, alkanol ammonium or metal cation; and
c) at least one C₂ to C₄ monoalkanolamide and/or dialkanolamide of at least one C₈ to C₂₄ carboxylic acid.

2. The cosmetic cleansing agent according to claim 1, including
a) the at least one anionic surfactant according to formula (I), in which
at least one of the groups R² to R⁵ denotes a methyl group, and the remaining groups denote a hydrogen atom;
R¹ denotes a linear or branched, saturated or unsaturated alkyl group having 8 to 18 carbon atoms; and
M⁺ denotes a sodium, potassium or ammonium ion;
b) at least one anionic surfactant according to formula (II), in which
R′ denotes a linear, saturated alkyl group having 8 to 18 carbon atoms;
n denotes a number from 1 to 8; and
X denotes a sodium, potassium or ammonium ion; and
c) at least one monoethanolamide of a C₈ to C₁₈ carboxylic acid.

3. The cosmetic cleansing agent according to claim 1_{;} wherein
a) at least one of Sodium Lauroyl Methyl Isethionate and Sodium Cocoyl Methyl Isethionate is included as the at least one first anionic surfactant;
b) Disodium Laureth Sulfosuccinate is included as the at least one second anionic surfactant; and
c) at least one of Cocamide MEA, Cocamide TAMPA or Lauramide MEA is included as the at least one C₂ to C₄ monoalkanolamide and/or dialkanolamide.

4. The cosmetic cleansing agent according to claim 1, wherein.
the percentage by weight of the at least one anionic surfactant according to formula (I) based the total weight of the composition is 1 to 20 wt. %;
the percentage by weight of the at least one anionic surfactant according to formula (II) based the total weight of the composition is 0.5 to 15 wt. %; and
the percentage by weight of the at least one C₂ to C₄ monoalkanolamide and/or dialkanolamide of at least one C₈ to C₂₄ carboxylic acid based the total weight of the composition is 0.1 to 5 wt. %.

5. The cosmetic cleansing agent according to claim 1, further including at least one conditioning active ingredient complex d), including at least one silicone compound and at least one cationic polymer.

6. The cosmetic cleansing agent according to claim 5, wherein the conditioning active ingredient complex (d) includes
at least one silicone compound of the following formula (III), 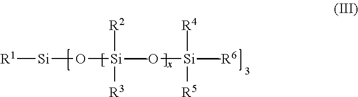
in which
R¹ to R⁵ independently of one another denote an aryl, alkyl or alkyl aryl group having 1 to 30 carbon atoms;
R⁶ denotes a group carrying at least one quaternary ammonium group; and
x denotes a number in the range from 2 to 200; and/or
at least one silicone compound of the following formula (IV), 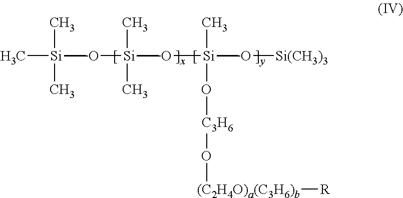
in which
the group R denotes a hydrogen atom, an alkyl group having 1 to 12 carbon atoms, an alkoxy group having 1 to 12 carbon atoms or a hydroxyl group, x denotes a number from 1 to 100;
y denotes a number from 1 to 20; and
a and b denote numbers from 0 to 50; and
at least one cationic polymer, selected from the group consisting of quatemized cellulose polymers, cationic guar derivatives and cationic polymers based on acrylic acid (derivatives).

7. The cosmetic cleansing agent according to claim 5, wherein the conditioning active ingredient complex (d) includes at least one silicone compound known by the INCI name Silicone Quaternium-22 and/or at least one silicone compound known by the INCI name PEG-12 Dimethicone, and further includes at least one cationic polymer selected from the group consisting of Polyquatemium-6, Polyquaternium-7, Polyquaternium-10 and Guar Hydroxypropyltrimonium Chloride.

8. The cosmetic cleansing agent according to claim 5, wherein the percentage by weight of the conditioning active ingredient complex d) based on the total weight of the composition is 0.1 to 10 wt. %.

9. The cosmetic cleansing agent according to claim 1, further including at least one amphoteric surfactant, the percentage by weight of the amphoteric surfactant based on the total weight of the composition being 0.1 to 15 wt. %.

10. The cosmetic cleansing agent according to claim 1, wherein the percentage by weight of sulfate group-containing surfactant based on the total weight of the composition is less than 0.5 wt. %.
